(19) United States
(12) Patent Application Publication
(10) Pub. No.: US 2025/0037872 A1
(43) Pub. Date: Jan. 30, 2025

(54) INFORMATION PROCESSING SYSTEM, INFORMATION PROCESSING METHOD, AND NON-TRANSITORY COMPUTER READABLE MEDIUM

(71) Applicant: NEC Corporation, Minato- ku, Tokyo (JP)

(72) Inventors: Yoshifumi Onishi, Tokyo (JP); Koichi Nihel, Tokyo (JP); Takanori Iwai, Tokyo (JP); Akira Yamauchi, Tokyo (JP); Koichi Kawashima, Tokyo (JP)

(73) Assignee: NEC Corporation, Minato- ku, Tokyo (JP)

(21) Appl. No.: 18/710,707

(22) PCT Filed: Dec. 2, 2021
(86) PCT No.: PCT/JP2021/044243

§ 371 Date: May 16, 2024

(51) Int. Cl. G16H 50/30; G06T 7/00

(52) U.S. Cl. CPC G16H 50/30; G06T 7/0012

(57) ABSTRACT

An information processing system includes: a registration unit that acquires, for each patient, a reference image obtained by photographing a patient and state information related to at least one of a physical condition state or a progress status of the patient at the time of photographing; a model generation unit that generates, for each patient, a physical condition estimation model for estimating a physical condition of the patient based on the reference image and the state information; a physical condition information generation unit that generates information related to the physical condition of the target patient by inputting a photographed image or predetermined state information of the target patient to the physical condition estimation model for the target patient; and an output control unit that outputs the information related to the physical condition of the target patient.

### TECHNICAL FIELD

The present disclosure relates to an information processing system, an information processing method, and a non-transitory computer readable medium, and more particularly, to an information processing system, an information processing method, and a non-transitory computer readable medium for estimating a physical condition of a patient.

### BACKGROUND ART

It is desired to monitor a physical condition of a patient after discharge and to cope with a case where the physical condition deteriorates or medicine is not effective at an early stage. For example, Patent Literature 1 discloses an estimation device that estimates a value of a physical condition of a subject by using skin cell image data of the subject as input data and using an estimation model learned by collecting skin cell image data of a plurality of persons and training data to be learned which are a set of skin image data and a physical condition.

In addition, for example, Patent Literature 2 discloses a system that determines whether or not a caregiver needs to be notified regarding deterioration of a patient's symptom based on an individual value corresponding to time in a sequence including a quantitative value of the patient's symptom.

Patent Literature 3 discloses a method for identifying a patient from tooth image data as image data of a body of the patient.

### CITATION LIST

### Patent Literature

Patent Literature 1: Japanese Unexamined Patent Application Publication No. 2020-085856
Patent Literature 2: Japanese Unexamined Patent Application Publication No. 2020-000871
Patent Literature 3: Japanese Unexamined Patent Application Publication No. 2020-108598

### SUMMARY OF INVENTION

### Technical Problem

Here, there is a need for a patient himself/herself or a person related to the patient to easily grasp a level of deterioration in physical condition and effectiveness of medicine without requiring special equipment. However, in Patent Literature 1 described above, a dedicated device for acquiring cell image data is required, and in Patent Literature 2 described above, a dedicated device for quantifying a symptom is required. Therefore, it is difficult to apply the present disclosure to a home or a simple clinic with insufficient equipment.

In addition, there is an individual difference in apparent reaction or complexion for deterioration in physical condition or pain, but the method described in Patent Literature 2 does not take into account the individual difference.

In view of the problems described above, an object of the present disclosure is to provide an information processing system, an information processing method, and a non-transitory computer readable medium capable of performing physical condition estimation personalized to an individual patient with simple equipment.

### Solution to Problem

An information processing system according to an aspect of the present disclosure includes:
registration means for acquiring, for each patient, a reference image obtained by photographing a patient and state information regarding at least one of a physical condition state or a progress status of the patient at a time of photographing;
model generation means for generating, for each patient, a physical condition estimation model for estimating a physical condition of the patient based on the reference image and the state information;
physical condition information generation means for generating information related to the physical condition of a target patient by inputting a photographed image or predetermined state information of the target patient to the physical condition estimation model for the target patient; and
output control means for outputting the information related to the physical condition of the target patient.

An information processing method according to an aspect of the present disclosure includes:
acquiring, for each patient, a reference image obtained by photographing a patient and state information regarding a physical condition state or a progress status of the patient at a time of photographing;
generating, for each patient, a physical condition estimation model for estimating a physical condition of the patient based on the reference image and the state information;
generating information related to the physical condition of a target patient by inputting a photographed image or predetermined state information of the target patient to the physical condition estimation model for the target patient; and
outputting the information related to the physical condition of the target patient.

A non-transitory computer readable medium according to an aspect of the present disclosure stores a program for causing a computer to execute processes of:
acquiring, for each patient, a reference image obtained by photographing a patient and state information regarding a physical condition state or a progress status of the patient at a time of photographing;
generating, for each patient, a physical condition estimation model for estimating a physical condition of the patient based on the reference image and the state information;
generating information related to the physical condition of a target patient by inputting a photographed image or predetermined state information of the target patient to the physical condition estimation model for the target patient; and
outputting the information related to the physical condition of the target patient.

### Advantageous Effects of Invention

According to the present disclosure, it is possible to provide an information processing system, an information processing method, and a non-transitory computer readable medium capable of performing physical condition estimation personalized to an individual patient with simple equipment.

### BRIEF DESCRIPTION OF DRAWINGS

FIG. 1 is a block diagram illustrating a configuration of an information processing system according to a first example embodiment.

FIG. 2 is a flowchart illustrating a flow of an information processing method according to the first example embodiment.

FIG. 3 is a block diagram illustrating an overall configuration of an information processing system according to a second example embodiment.

FIG. 4 is a block diagram illustrating a configuration of a server according to the second example embodiment.

FIG. 5 is a sequence diagram illustrating an example of a flow of a registration process according to the second example embodiment.

FIG. 6 is a view illustrating an example of display on a hospital terminal according to the second example embodiment.

FIG. 7 is a sequence diagram illustrating an example of a flow of an output process for physical condition related information according to the second example embodiment.

FIG. 8 is a view illustrating an example of display on a patient terminal according to the second example embodiment.

FIG. 9 is a view illustrating an example of display on the patient terminal according to the second example embodiment.

FIG. 10 is a view illustrating an example of display on the patient terminal according to the second example embodiment.

FIG. 11 is a block diagram illustrating a configuration of a server according to a third example embodiment.

FIG. 12 is a sequence diagram illustrating an example of a flow of an output process for physical condition related information according to the third example embodiment.

FIG. 13 is a view illustrating an example of display on a patient terminal according to the third example embodiment.

FIG. 14 is a diagram illustrating a configuration example of a computer.

### EXAMPLE EMBODIMENT

Hereinafter, example embodiments of the present disclosure will be described in detail with reference to the drawings. In the drawings, the same or corresponding elements are denoted by the same reference numerals, and redundant description is omitted as necessary for clear description.

### First Example Embodiment

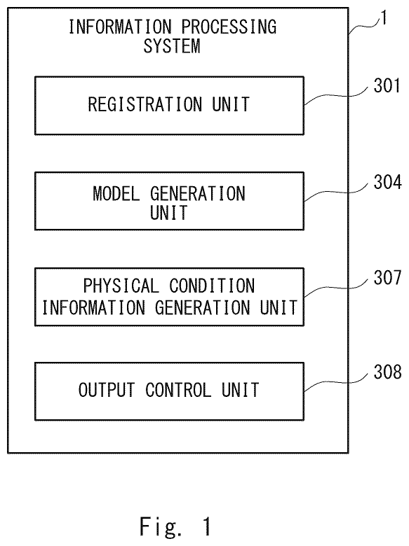

First, a first example embodiment of the present disclosure will be described. FIG. 1 is a block diagram illustrating a configuration of an information processing system 1 according to the first example embodiment. The information processing system 1 is a computer system including one computer device or a plurality of computer devices for estimating a state related to a physical condition of a patient. Hereinafter, the term “state” refers to a physical condition state or a progress status.

The information processing system 1 includes a registration unit 301, a model generation unit 304, a physical condition information generation unit 307, and an output control unit 308.

The registration unit 301 is also referred to as registration means. The registration unit 301 acquires, for each patient, a reference image obtained by photographing a patient and patient state information at the time of photographing the reference image.

The reference image is a photographed image obtained by photographing the whole or a part of a body of the patient with a camera. The reference image may be a still image or a moving image. Hereinafter, a region to be photographed, that is, a region corresponding to an image area included in the reference image, may be referred to as a target region. The target region may include a region where a complexion or expression of the patient can be detected, or may include a region used by medical personnel for case determination for the patient. Examples of the target region may include a face, eyelid, arm, leg, or neck. In a case where the camera photographs the entire body of the patient, the target region is the entire body.

The time of photographing may be a photographing time point or any time point within a predetermined period from the photographing time point.

The state information is, for example, information regarding at least one of a physical condition state or a progress status. The physical condition state may indicate a state level related to the physical condition, such as a level of a symptom, a level of progression of a disease, a level of recovery of a disease, a level of injury or a level of consciousness. As an example, the state level may include a state in which the physical condition is good, a state in which the physical condition is normal, a state in which the physical condition is bad, and the like. In addition, the physical condition state may be an effect of medicine or treatment for injuries or diseases.

Here, the progress status may be a patient's status at the time of photographing with a certain status as a starting event. Examples of information regarding the progress status may include information indicating that the photographing is performed at the time of admission, on the n-th day of hospitalization (n is a natural number), or at the time of discharge. In this case, the information regarding the progress status indicates a status suggesting the progress of the disease, and the starting event is admission. Furthermore, as an example, the information regarding the progress status may include information regarding an elapsed time since taking medicine or undergoing treatment. In this case, the information regarding the progress status indicates a status suggesting the progress of the physical condition after taking medicine or treatment, and the starting event is taking medicine or undergoing treatment.

The state information may be information in which the physical condition state and the progress status are combined.

Then, the registration unit 301 registers a set of the reference image and the state information of the patient in a database (DB) (not illustrated in the drawings).

The model generation unit 304 is also referred to as model generation means. The model generation unit 304 generates, for each patient, a physical condition estimation model for estimating the physical condition of the patient based on a set of the reference image and the state information acquired by the registration unit 301 and registered in the DB. Details of an input and an output of the physical condition estimation model will be described later.

The physical condition information generation unit 307 is also referred to as physical condition information generation means. The physical condition information generation unit 307 generates information related to the physical condition of the target patient by using the physical condition estimation model for the target patient. The information related to the physical condition of the target patient is also referred to as physical condition related information. The physical condition related information may be various information related to the physical condition, and may be, for example, the following (Case 1) or (Case 2).

(Case 1) The physical condition related information may be estimated state information which is state information estimated from the physical condition estimation model, or may be information generated based on the estimated state information. The estimated state information may indicate at least one of the current physical condition state or the progress status of the patient. In a case where the estimated state information indicates the current physical condition state of the patient, the information generated based on the estimated state information may be, for example, information indicating which state (for example, at the time of admission, on the n-th day of hospitalization, or at the time of discharge) in the past the current physical condition state of the patient is similar to. Furthermore, in the case described above, the information generated based on the estimated state information may be information indicating whether or not a patient P needs to be examined by a doctor.

In case of (Case 1), the physical condition estimation model described above is a first physical condition estimation model that receives the photographed image of the target region of the patient as an input and outputs the estimated state information of the patient. The input photographed image may be a still image or a moving image. The first physical condition estimation model may include a convolutional neural network (CNN). In addition, the first physical condition estimation model may be a regression model using a regression equation indicating a relationship between a feature of the target region of the patient included in the photographed image or a change amount thereof and the state at the time of photographing. The reference image and the state information registered in the DB are used to generate the first physical condition estimation model. Then, the physical condition information generation unit 307 is configured to use the estimated state information that is an output result of the first physical condition estimation model as the physical condition related information or generate the physical condition related information based on the estimated state information.

(Case 2) The physical condition related information may be a simulation image serving as a guide for estimating the physical condition. The simulation image may be used in applications where the patient, a family member of the patient, or other person related to the patient performs comparison with the current appearance of the patient to determine whether or not the patient needs to be examined by a doctor. In addition, the simulation image may be used for comparison with the current appearance of the patient by a doctor or a medical staff of a hospital in order to simply grasp an effect of medicine or treatment, for example, an effect of anesthesia.

In case of (Case 2), the physical condition estimation model described above is a second physical condition estimation model that outputs the simulation image of the target region of the patient with predetermined state information as an input. The second physical condition estimation model may include a generative adversarial network (GAN) or a decoder-type network of a convolutional neural network (CNN). The physical condition information generation unit 307 can obtain the simulation image as the physical condition related information by inputting the predetermined state information to the second physical condition estimation model. The reference image and the state information registered in the DB are used to generate the second physical condition estimation model.

The output control unit 308 is also referred to as output control means. The output control unit 308 outputs the physical condition related information of the target patient generated by the physical condition information generation unit 307. The output may be transmission, transmission to a predetermined display device for display, or transmission to a predetermined voice output device for output in a case where the physical condition related information is text data. For example, the output control unit 308 may transmit the physical condition related information of the target patient to a terminal used by the target patient or a family member of the target patient. Furthermore, for example, the output control unit 308 may transmit the physical condition related information of the target patient to a terminal managed by a hospital or a designated hospital at the time of admission of the target patient. Then, the terminal that has received the physical condition related information may display the physical condition related information of the target patient, and may output the physical condition related information of the target patient by voice in a case where the physical condition related information is text data.

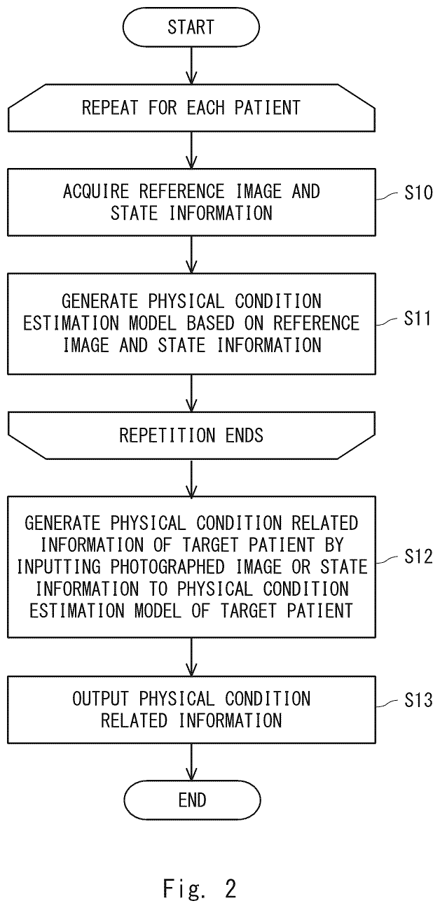

FIG. 2 is a flowchart illustrating a flow of an information processing method according to the first example embodiment. First, the information processing system 1 repeats processes of S10 and S11 for each patient. In S10, the registration unit 301 of the information processing system 1 acquires a reference image and state information of a certain patient. Then, the registration unit 301 accumulates a set of the reference image and the state information of the patient in the DB. Next, in S11, the model generation unit 304 generates a physical condition estimation model based on the reference image and the state information registered in the DB.

Next, in S12, the physical condition information generation unit 307 generates physical condition related information of a target patient by inputting a photographed image of a target region of the target patient or predetermined state information to the physical condition estimation model for the target patient. Then, in S13, the output control unit 308 outputs the physical condition related information to a destination terminal.

As described above, according to the first example embodiment, the information processing system 1 generates and outputs the physical condition related information of the patient by using the physical condition estimation model generated for each patient based on the reference image and the state information. Therefore, the information processing system 1 can generate the physical condition estimation model personalized to an individual patient by simple equipment such as a camera. In addition, the information processing system 1 generates the physical condition related information by inputting the photographed image or the state information. Therefore, the information processing system 1 can provide the physical condition related information personalized to an individual patient by simple equipment such as a camera or an input device. In particular, at home or in a simple clinic with insufficient equipment, it is possible to easily grasp the physical condition of a patient and to cope with a case where the physical condition deteriorates or medicine is not effective at an early stage. Furthermore, the information processing system 1 is useful not only at home or in a simple clinic but also in a case where a doctor or a medical staff in a hospital simply grasps the physical condition of a patient or the effectiveness of medicine.

### Second Example Embodiment

Next, a second example embodiment of the present disclosure will be described. The second example embodiment is a specific example in which physical condition related information is (Case 1) described above. That is, in the second example embodiment, a physical condition estimation model is a first physical condition estimation model that receives a photographed image of a target region of a patient as an input and outputs estimated state information of the patient.

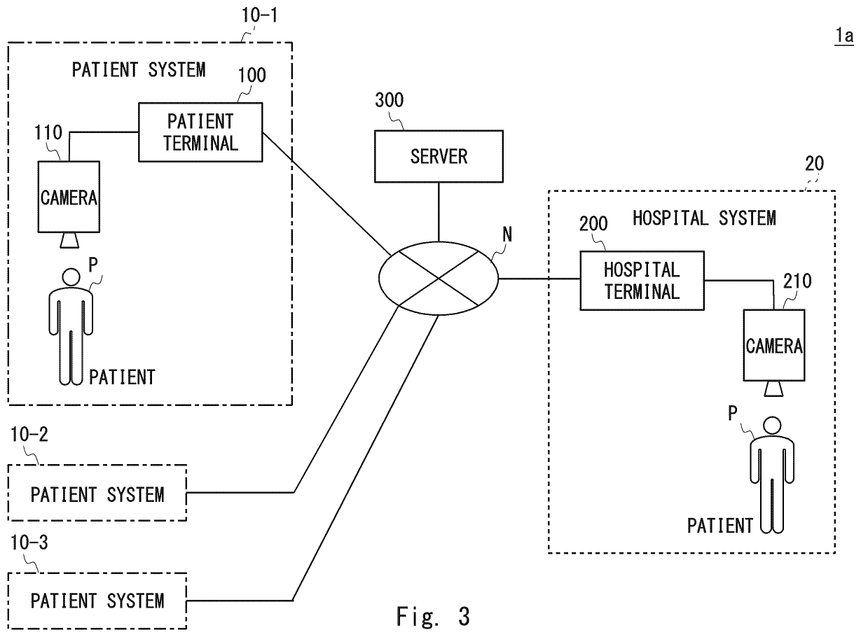

FIG. 3 is a block diagram illustrating an overall configuration of an information processing system 1a according to the second example embodiment. The information processing system 1a is an example of the information processing system 1 described above. The information processing system 1a includes a plurality of patient systems 10-1, 10-2, and 10-3, a hospital system 20, and an information processing apparatus (hereinafter, referred to as a server) 300. Each device and system are connected to a wired or wireless network N. The number of patient systems 10 is an example and is not limited thereto. Hereinafter, a family member of a patient P, a medical staff at a simple clinic, or other doctors and medical staffs who want to grasp the physical condition related information are collectively referred to as associated persons.

### (Hospital System 20)

The hospital system 20 is a computer system of a hospital in which the patient P is hospitalized or a hospital associated with the patient P. The hospital system 20 acquires a reference image of the patient P who is hospitalized or examined, and transmits the reference image to the server 300 in association with the physical condition related information.

Specifically, the hospital system 20 includes a camera 210 and a hospital terminal 200.

The camera 210 is provided in the hospital. For example, the camera 210 is installed in a room of the patient P who is hospitalized, a consultation room, or an examination room. As an example, the camera 210 photographs a target region of the patient P lying on a bed in the room. As an example, the camera 210 photographs the target region of the patient P being examined or treated. In addition, as an example, the camera 210 photographs the target region of the patient P being examined in an examination device. The camera 210 is connected to the hospital terminal 200, and transmits the reference image generated by photographing to the hospital terminal 200.

The hospital terminal 200 is an information terminal provided in the hospital or an information terminal managed by a doctor or other staff of the hospital. For example, the hospital terminal 200 is a personal computer, a smartphone, or a tablet terminal. The hospital terminal 200 is connected to the network N. The hospital terminal 200 acquires the reference image of the patient P from the camera 210. In addition, the hospital terminal 200 acquires state information corresponding to the reference image. For example, the hospital terminal 200 acquires the state information by receiving an input of the state information in a period close to a photographing period of the reference image from a doctor or staff of the hospital. Furthermore, for example, the hospital terminal 200 can read medical record information, which is information described in a medical record of the patient P, and may acquire the state information by extracting the state information at a time point close to a photographing time point of the reference image from the medical record information of the patient P. The time point close to the photographing time point may refer to an arbitrary time point within a predetermined period from the photographing time point. Then, the hospital terminal 200 transmits an image registration request including information in which the reference image and the state information are associated with each other to the server 300 via the network N for each patient.

### (Patient System 10)

The patient system 10 is a computer system of the home of the patient P. Alternatively, the patient system 10 may be a computer system in a simple clinic or other remote facility. For example, the patient system 10 transmits a photographed image of the target region of the patient P after discharge or after examination in the hospital to the server 300, and receives the physical condition related information generated based on the photographed image from the server 300.

Specifically, the patient system 10 includes a camera 110 and a patient terminal 100.

The camera 110 is connected to the patient terminal 100. The camera 110 photographs the target region of the patient P after discharge at a location remote from the hospital in response to an operation on an application by the patient P or a person related to the patient P or under the automatic control of the application. The camera 110 transmits a photographed image generated by photographing to the patient terminal 100. The camera 110 may be integrally mounted on the patient terminal 100.

The patient terminal 100 is an information terminal used by the patient P or a person related to the patient P. For example, the patient terminal 100 is a personal computer, a smartphone, or a tablet terminal. The patient terminal 100 is connected to the network N. The patient terminal 100 activates the application and acquires, from the camera 110, the photographed image of the target region of the patient P after discharge or after examination in the hospital. Then, the patient terminal 100 transmits an output request for the physical condition related information including the photographed image and a patient ID to the server 300. The patient ID is information for identifying a patient, and may be a patient name, a registration card number, or other identification numbers.

The patient terminal 100 receives the physical condition related information of the patient P from the server 300 that has received the output request. Then, the patient terminal 100 displays the physical condition related information on a display unit (not illustrated in the drawings) or outputs the physical condition related information by voice through a voice output unit (not illustrated in the drawings).

### (Server 300)

The server 300 is a computer device for estimating a physical condition of each patient. In a case where the image registration request is received from the hospital terminal 200, the server 300 generates data to be learned (hereinafter, also referred to as “training data”) for the patient P in which the reference image and the state information are associated with each other. Then, the server 300 generates the first physical condition estimation model for estimating the state of the patient P based on the training data for the patient P. Then, the server 300 generates the first physical condition estimation model for each patient.

In addition, when the output request for the physical condition related information is received from the patient terminal 100, the server 300 generates the physical condition related information of the target patient by using the first physical condition estimation model for the patient P. Then, the server 300 transmits the physical condition related information to the patient terminal 100.

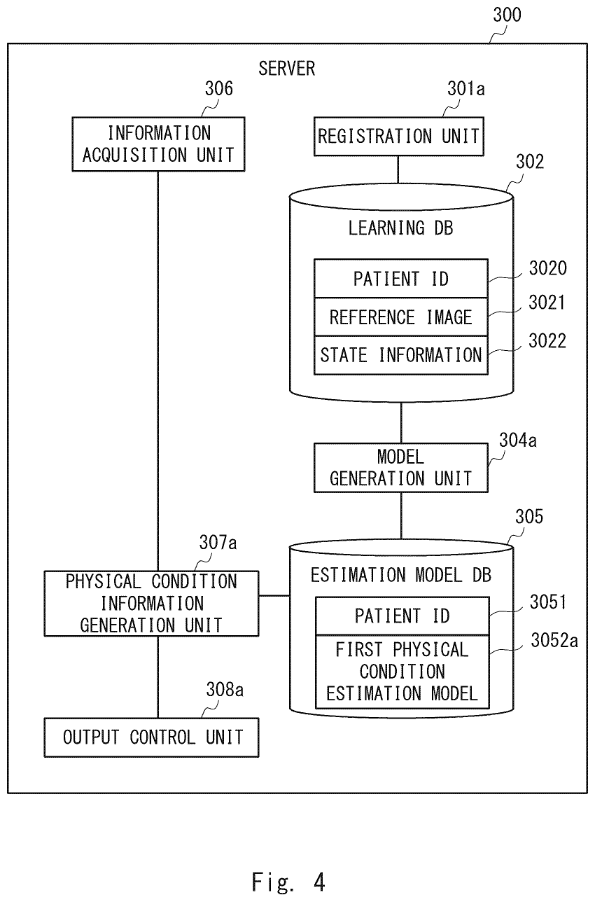

FIG. 4 is a block diagram illustrating a configuration of the server 300 according to the second example embodiment. The server 300 includes a registration unit 301a, a learning (machine learning) DB 302, a model generation unit 304a, an estimation model DB 305, an information acquisition unit 306, a physical condition information generation unit 307a, and an output control unit 308a.

The registration unit 301a is an example of the registration unit 301 described above.

In a case where a registration request (patient registration request) for the patient P is received from the hospital terminal 200 for each patient, the registration unit 301a determines the target region of the patient P. For example, in a case where a target region designated by a doctor or staff of the hospital is acquired from the hospital terminal 200, the registration unit 301a may determine the designated target region as the target region of the patient P. Furthermore, for example, the registration unit 301a may acquire the medical record information of the patient P from the hospital terminal 200 and determine the target region based on the medical record information of the patient P. As an example, the registration unit 301a may determine the target region corresponding to a disease name of the patient P included in the medical record information as the target region of the patient P. In the case of utilizing the medical record information together, it is possible to save a doctor or medical staff the trouble of inputting.

In addition, in a case where the image registration request is received from the hospital terminal 200, the registration unit 301a generates training data by labeling the state information included in the image registration request on the reference image included in the image registration request. In the second example embodiment, the state information may be a value obtained by quantifying the state, a vector, or a matrix, or a class name to which the state belongs. The registration unit 301a registers the training data in the learning DB 302 in association with the patient ID.

The learning DB 302 is a storage device that stores training data for a plurality of patients. The learning DB 302 stores a patient ID 3020, a reference image 3021, and state information 3022 in association with each other. The reference image 3021 and the state information 3022 are training data.

The model generation unit 304a is an example of the model generation unit 304 described above. The model generation unit 304a learns (or performs machine learning for) the first physical condition estimation model for each patient by using the training data included in the learning DB 302. Learning the first physical condition estimation model may be optimizing parameters of the first physical condition estimation model. For example, the model generation unit 304a may learn all the parameters of the first physical condition estimation model determined in advance for the patient P by using the training data for the patient P. Alternatively, the model generation unit 304a may learn some parameters of the first physical condition estimation model determined in advance for the patient P by using the training data for the patient P. As a result, the model generation unit 304a can generate the first physical condition estimation model for each patient. Then, the model generation unit 304a stores the first physical condition estimation model for each patient in the estimation model DB 305.

The estimation model DB 305 is a storage device that stores the first physical condition estimation model for each patient. Specifically, the estimation model DB 305 stores a patient ID 3051 and a first physical condition estimation model 3052a in association with each other.

The information acquisition unit 306 is also referred to as information acquisition means. The information acquisition unit 306 receives the output request for the physical condition related information including the photographed image of the target region of the patient P from the patient terminal 100. As a result, the information acquisition unit 306 acquires the photographed image of the target region of the patient P. Then, the information acquisition unit 306 supplies, to the physical condition information generation unit 307a, the acquired photographed image and the patient ID associated with the patient terminal 100 that is an output request source.

The physical condition information generation unit 307a is an example of the physical condition information generation unit 307 described above.

The physical condition information generation unit 307a reads the first physical condition estimation model associated with the patient ID acquired from the information acquisition unit 306 by referring to the estimation model DB 305. Then, the physical condition information generation unit 307a generates the physical condition related information from the photographed image acquired from the information acquisition unit 306 by using the first physical condition estimation model. Specifically, the physical condition information generation unit 307a inputs the photographed image to the first physical condition estimation model, and obtains the estimated state information as an output result. Then, the physical condition information generation unit 307a sets the output result as the physical condition related information or generates the physical condition related information based on the output result.

As an example, in a case where the state information indicates a state level related to the physical condition, the estimated state information which is the output result of the first physical condition estimation model may indicate the current state level related to the physical condition of the patient P. In a case where the state information further contains a status of the patient P at the time of photographing, the estimated state information which is the output result of the first physical condition estimation model may indicate which status in the past the current state of the patient P is similar to.

Then, in a case where the physical condition related information indicates whether or not the patient P needs to be examined by a doctor, the physical condition information generation unit 307a may determine whether or not the patient P needs to be examined by a doctor based on the current state of the patient P. For example, in a case where the current state level of the patient P is equal to or lower than a state level in a predetermined status (for example, at the time of admission), the physical condition information generation unit 307a may generate the physical condition related information indicating that the patient P needs to be examined by a doctor. On the other hand, in a case where the current state level of the patient P is higher than the state level in the predetermined status, the physical condition information generation unit 307a may generate the physical condition related information indicating that the patient P does not need to be examined by a doctor.

In addition to or instead of the current state level of the patient P, the physical condition information generation unit 307a may determine whether or not the patient P needs to be examined by a doctor based on a progress status of the patient P. For example, in a case where the state level of the patient P decreases at a speed equal to or higher than a predetermined reference speed, the physical condition information generation unit 307a may generate the physical condition related information indicating that the patient P needs to be examined by a doctor. Furthermore, for example, in a case where the state level of the patient P does not change in a predetermined period and the current state level of the patient P is equal to or lower than the state level in the predetermined status, the physical condition information generation unit 307a may generate the physical condition related information indicating that the patient P needs to be examined by a doctor.

The output control unit 308a is an example of the output control unit 308 described above. The output control unit 308a transmits the physical condition related information of the patient P to the patient terminal 100 of the patient P and displays the physical condition related information.

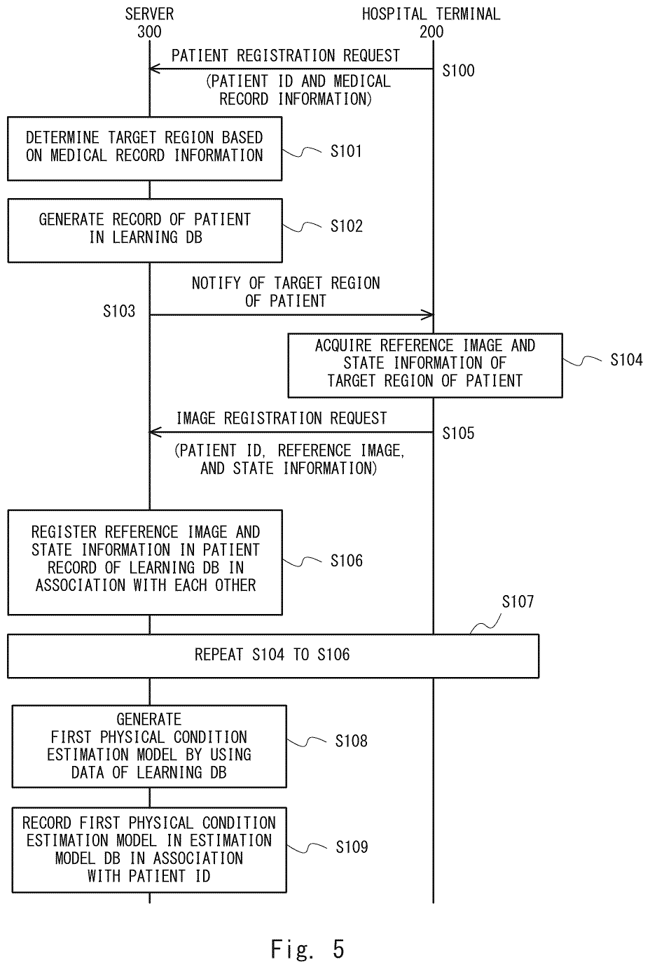

FIG. 5 is a sequence diagram illustrating an example of a flow of a registration process according to the second example embodiment. First, the hospital terminal 200 transmits a patient registration request including a patient ID and medical record information to the server 300 (S100).

The registration unit 301a of the server 300 determines a target region of a patient P based on the medical record information included in the patient registration request (S101). Then, the registration unit 301a generates a record of the patient P in the learning DB 302 (S102). Specifically, the registration unit 301a generates a record corresponding to the patient ID of the patient P in the learning DB 302. The registration unit 301a notifies the hospital terminal 200 of the target region of the patient P (S103).

Next, the hospital terminal 200 acquires a reference image of the target region of the patient P from the camera 210, and acquires state information based on the medical record information (S104). Then, the hospital terminal 200 transmits an image registration request to the server 300 (S105). The image registration request may include the patient ID, the reference image of the target region of the patient P, and the state information at the time of photographing the reference image.

Next, the registration unit 301a of the server 300 registers the reference image and the state information as training data in the record corresponding to the patient ID of the patient P in the learning DB 302 in association with each other by labeling or the like (S106).

Then, the information processing system 1a repeats the processes of S104 to S106, and ends the repetition in a case where a predetermined condition is satisfied (S107). For example, in a case where the processes of S104 to S106 are repeated a predetermined number of times, the information processing system 1a may end the repetition. Alternatively, the information processing system 1a may end the repetition in a case where a necessary amount or more of training data is stored in the learning DB 302. Alternatively, the information processing system 1a may end the repetition in a case where a necessary amount or more of training data corresponding to a predetermined state level or status is stored in the learning DB 302. The number of repetitions or the necessary amount of training data is the number of times or amount necessary for ensuring the accuracy of a first physical condition estimation model. The number of repetitions or the necessary amount of training data may be changed depending on the type of the target region, the state information, or the physical condition related information.

Next, the model generation unit 304a of the server 300 generates the first physical condition estimation model for the patient P by using the training data of the learning DB 302 in response to the end of the repetition (S108). Then, the model generation unit 304a of the server 300 stores the first physical condition estimation model in the estimation model DB 305 in association with the patient ID of the patient P (S109).

The server 300 can store the first physical condition estimation model personalized for each patient in the learning DB 302 by repeating the flow described above for each patient.

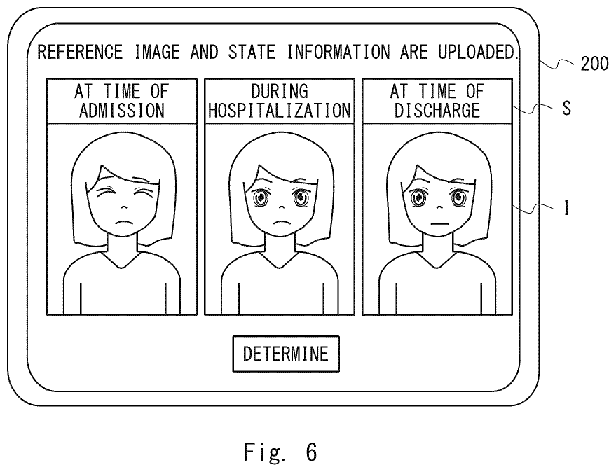

Here, when the hospital terminal 200 transmits the image registration request in S105, a display screen illustrated in FIG. 6 may be displayed on a display unit of the hospital terminal 200. FIG. 6 is a view illustrating an example of display on the hospital terminal 200 according to the second example embodiment. In this example, the target region is a face. For example, from this screen, a doctor or staff of the hospital can upload a reference image of the face of the patient P for each of the state information of “at the time of admission”, “during hospitalization”, and “at the time of discharge”. According to the uploaded reference images, as time passes, the expression of the patient P changes from an expression when the physical condition is remarkably bad to an expression when the physical condition recovers, as indicated by “at the time of admission”, “during hospitalization”, and “at the time of discharge”. An operation area for determining a combination of the reference image and the state information uploaded by a doctor or staff of the hospital is displayed on the display unit of the hospital terminal 200. When a doctor or staff of the hospital taps the operation area, the hospital terminal 200 can transmit the image registration request to the server 300.

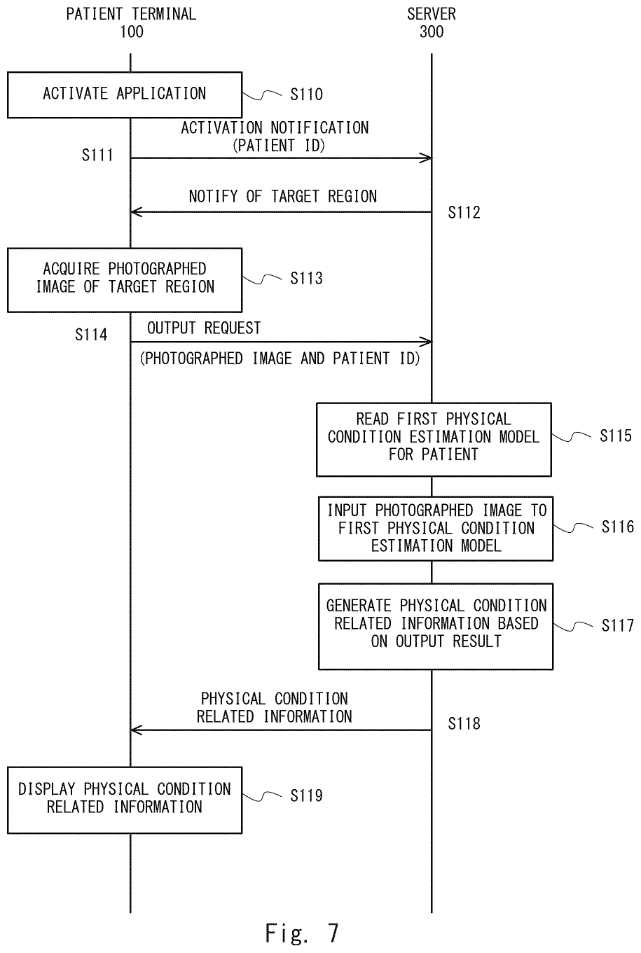

FIG. 7 is a sequence diagram illustrating an example of a flow of an output process for the physical condition related information according to the second example embodiment. First, the patient terminal 100 activates an application for browsing physical condition related information (S110). In response to activating the application, the patient terminal 100 transmits an activation notification to the server 300 (S111). The activation notification may include a patient ID of a patient P.

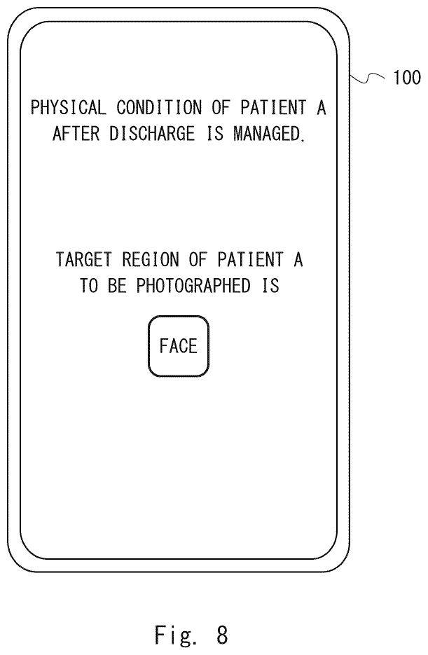

The server 300 that has received the activation notification notifies the patient terminal 100 of a target region (S112). A display screen of the patient terminal 100 in the notification of the target region is illustrated in FIG. 8. FIG. 8 is a view illustrating an example of display on the patient terminal 100 according to the second example embodiment. For example, the display unit of the patient terminal 100 displays a message such as “The physical condition of Patient A after discharge is managed. The target region of Patient A to be photographed is “face””.

Returning to FIG. 7, the description will be continued. The patient terminal 100 that has received the notification of the target region acquires a photographed image obtained by photographing a current target region of the patient P from the camera 110 (S113). Then, the patient terminal 100 transmits an output request for the physical condition related information to the server 300 (S114). The output request may include the photographed image of the target region of the patient P and the patient ID.

As a result, the information acquisition unit 306 of the server 300 acquires the photographed image of the target region of the patient P and the patient ID. Then, the physical condition information generation unit 307a of the server 300 reads the first physical condition estimation model associated with the patient ID in the estimation model DB 305 by referring to the estimation model DB 305 (S115). Next, the physical condition information generation unit 307a inputs the photographed image to the first physical condition estimation model (S116). Next, the physical condition information generation unit 307a generates the physical condition related information based on an output result of the first physical condition estimation model (S117). The output control unit 308a of the server 300 transmits the physical condition related information to the patient terminal 100 that is an output request source (S118).

Then, the patient terminal 100 receives the physical condition related information and displays the physical condition related information on the display unit (S119).

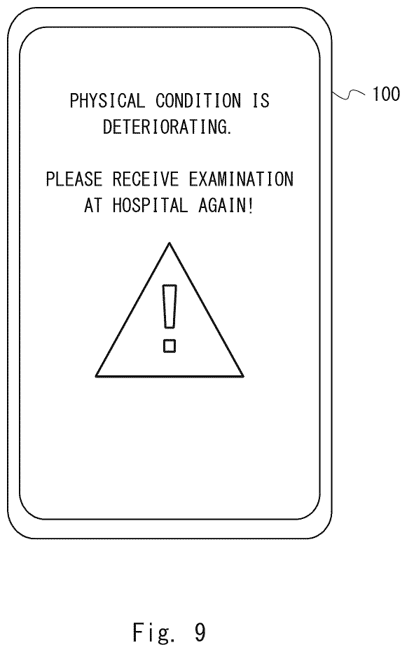

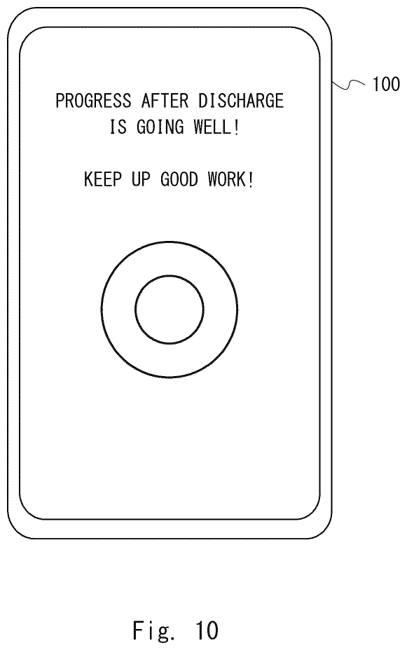

FIGS. 9 and 10 are views illustrating an example of display on the patient terminal 100 according to the second example embodiment. In FIG. 9, for example, a message prompting a re-examination at the hospital is displayed as the physical condition related information together with a message indicating that the physical condition deteriorates on the display unit of the patient terminal 100. By viewing this screen, the patient P or a person associated with the patient P can easily monitor the physical condition of the patient P and cope with a case where the physical condition deteriorates or medicine is not effective at an early stage.

In FIG. 10, for example, a message indicating that the progress after discharge is favorable is displayed on the display unit of the patient terminal 100. The patient P or a person associated with the patient P can grasp that it is not necessary to visit the hospital after discharge at the present stage by browsing the screen. Therefore, it is possible to avoid a case where the user feels anxious about whether or not to receive an examination or goes to a hospital unnecessarily. In addition, a message for encouraging the patient P is displayed on the display unit of the patient terminal 100, so that the psychological burden on the patient P may be reduced.

As described above, according to the second example embodiment, it is possible to perform physical condition estimation personalized to an individual patient by simple equipment such as a camera. As a result, the patient P and a person associated with the patient P can easily confirm an appropriate timing for the patient P to receive an examination after discharge from the hospital, and thus, it is possible to eliminate the excessive anxiety of the patient P after discharge from the hospital. For the hospital, it is possible to avoid attendance for an unnecessary examination, and the patient P can come to receive an examination at an appropriate timing when the physical condition deteriorates, so that it is possible to safely shorten a hospitalization period of the patient P. This enables efficient hospital management.

### Third Example Embodiment

Next, a third example embodiment of the present disclosure will be described. The third example embodiment is a specific example in which physical condition related information is (Case 2) described above. That is, in the third example embodiment, a physical condition estimation model is a second physical condition estimation model that receives predetermined state information as an input and outputs a simulation image of a target region of a patient.

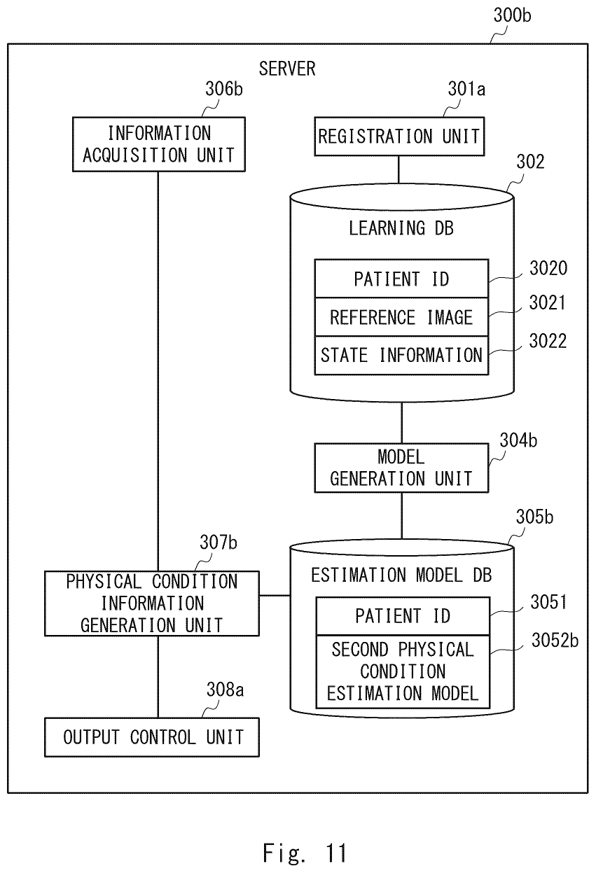

FIG. 11 is a block diagram illustrating a configuration of a server 300b according to the third example embodiment. The server 300b includes a model generation unit 304b, an estimation model DB 305b, an information acquisition unit 306b, and a physical condition information generation unit 307b instead of the model generation unit 304a, the estimation model DB 305, the information acquisition unit 306, and the physical condition information generation unit 307a.

The model generation unit 304b is an example of the model generation unit 304 described above. The model generation unit 304b learns (or performs machine learning for) the second physical condition estimation model for each patient by using training data included in a learning DB 302. Learning the second physical condition estimation model may be optimizing parameters of the second physical condition estimation model. For example, the model generation unit 304b may learn all the parameters of the second physical condition estimation model determined in advance for a patient P by using the training data of the patient P. The model generation unit 304b may learn some parameters of the second physical condition estimation model determined in advance for the patient P by using the training data of the patient P. As a result, the model generation unit 304b can generate the second physical condition estimation model for each patient. Then, the model generation unit 304b stores the second physical condition estimation model for each patient in the estimation model DB 305b.

The estimation model DB 305b is a storage device that stores the second physical condition estimation model for each patient. Specifically, the estimation model DB 305b stores a patient ID 3051 and a second physical condition estimation model 3052b in association with each other.

The information acquisition unit 306b is also referred to as information acquisition means. The information acquisition unit 306b receives an output request for the physical condition related information including the patient ID from a patient terminal 100. As a result, the information acquisition unit 306b acquires the patient ID of the patient P. Then, the information acquisition unit 306 supplies the patient ID to the physical condition information generation unit 307b.

The physical condition information generation unit 307b is an example of the physical condition information generation unit 307 described above. The physical condition information generation unit 307b reads the second physical condition estimation model associated with the patient ID acquired from the information acquisition unit 306b by referring to the estimation model DB 305b. Then, the physical condition information generation unit 307b inputs predetermined state information to the second physical condition estimation model, and obtains a simulation image of a target region of the patient P corresponding to the state information as an output result. Then, the physical condition information generation unit 307b sets the output result as the physical condition related information. Then, the physical condition information generation unit 307b supplies the physical condition related information to an output control unit 308.

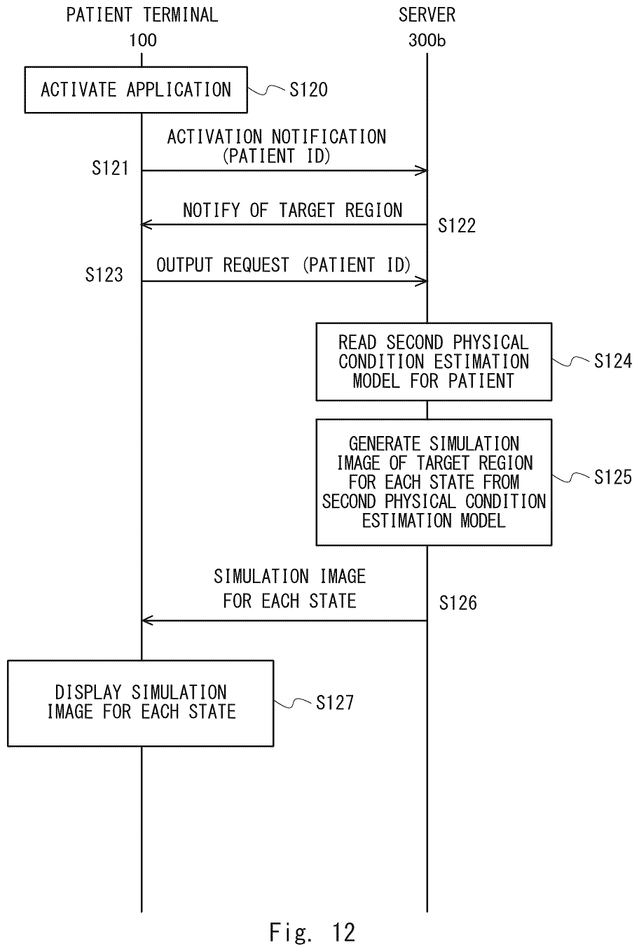

FIG. 12 is a sequence diagram illustrating an example of a flow of an output process for the physical condition related information according to the third example embodiment. First, S120 to S122 similar to S110 to S112 are performed. Next, in S123, the patient terminal 100 that has received a notification of a target region transmits an output request for physical condition related information to the server 300b. The output request may include a patient ID.

As a result, the information acquisition unit 306b of the server 300b acquires the patient ID. Then, the physical condition information generation unit 307b of the server 300b reads a second physical condition estimation model associated with the patient ID in the estimation model DB 305b by referring to the estimation model DB 305b (S124). Next, the physical condition information generation unit 307b generates a simulation image of a target region of a patient P for each state by inputting predetermined state information to the second physical condition estimation model (S125). Next, the output control unit 308b transmits the simulation image of the target region of the patient P for each state as the physical condition related information to the patient terminal 100 that is an output request source (S126).

Then, the patient terminal 100 receives the physical condition related information and displays the physical condition related information on the display unit (S127).

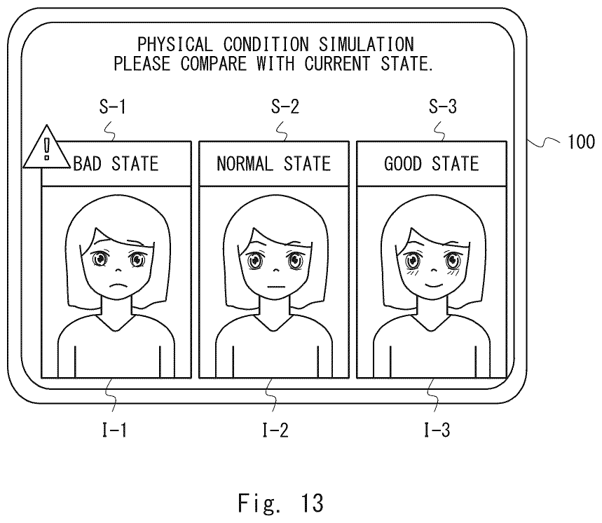

FIG. 13 is a view illustrating an example of display on the patient terminal 100 according to the third example embodiment. In FIG. 13, the predetermined state information that is an input of the second physical condition estimation model is a bad state (state S-1), a normal state (state S-2), and a good state (state S-3). For example, a simulation image I-1 corresponding to the state S-1, a simulation image I-2 corresponding to the state S-2, and a simulation image I-3 corresponding to the state S-3 are displayed on the display unit of the patient terminal 100 in association with the respective pieces of state information. The patient P or a person associated with the patient P can easily grasp a physical condition of the patient P by comparing the simulation image with the current target region of the patient P. Therefore, the patient P or a person associated with the patient P can easily monitor the physical condition of the patient P and can cope with a case where the physical condition deteriorates or medicine is not effective at an early stage.

Therefore, according to the third example embodiment, the same effects as those of the second example embodiment can be obtained.

In the example embodiments described above, the configuration of the hardware has been described, but the present disclosure is not limited thereto. In the present disclosure, any process can also be implemented by causing a processor to execute a computer program.

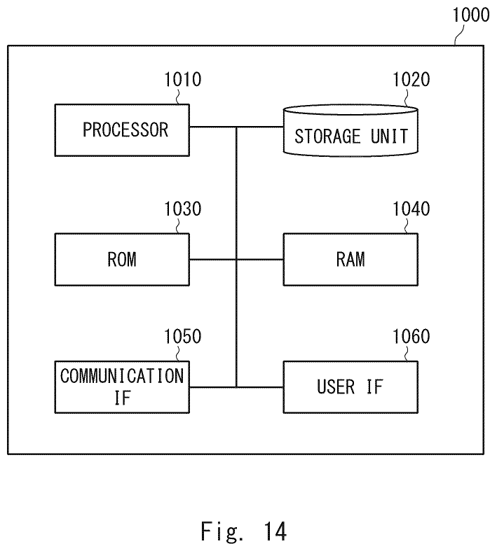

FIG. 14 is a diagram illustrating a configuration example of a computer used as the patient terminal 100, the hospital terminal 200, or the server 300. A computer 1000 includes a processor 1010, a storage unit 1020, a read only memory (ROM) 1030, a random access memory (RAM) 1040, a communication interface (IF) 1050, and a user interface 1060.

The communication interface 1050 is an interface for connecting the computer 1000 to a communication network through wired communication means, wireless communication means, or the like. The user interface 1060 includes, for example, a display unit such as a display. In addition, the user interface 1060 also includes an input unit such as a keyboard, a mouse, or a touch panel. Note that the user interface 1060 is not essential particularly for the server 300.

The storage unit 1020 is an auxiliary storage device that can hold various types of data. The storage unit 1020 does not necessarily have to be a part of the computer 1000, but may be an external storage device, or a cloud storage connected to the computer 1000 via a network.

The ROM 1030 is a non-volatile storage device. For example, a semiconductor storage device such as a flash memory having a relatively small capacity is used for the ROM 1030. A program that is executed by the processor 1010 can be stored in the storage unit 1020 or the ROM 1030. The storage unit 1020 or ROM 1030 stores, for example, various programs for implementing the function of each unit in the server.

In the example described above, the program includes a group of commands (or software codes) for causing the computer to perform one or more functions described in the example embodiments, when read by the computer. The program may be stored in a non-transitory computer readable medium or a tangible storage medium. As an example and not by way of limitation, the computer readable medium or the tangible storage medium includes a random-access memory (RAM), a read-only memory (ROM), a flash memory, a solid-state drive (SSD) or any other memory technology, a CD-ROM, a digital versatile disc (DVD), a Blu-ray (registered trademark) disc or any other optical disk storage, a magnetic cassette, a magnetic tape, a magnetic disk storage, and any other magnetic storage device. The program may be transmitted on a transitory computer readable medium or a communication medium. As an example and not by way of limitation, the transitory computer readable medium or the communication medium includes propagated signals in electrical, optical, acoustic, or any other form.

The RAM 1040 is a volatile storage device. As the RAM 1040, various types of semiconductor memory devices such as a dynamic random access memory (DRAM) or a static random access memory (SRAM) can be used. The RAM 1040 can be used as an internal buffer for temporarily storing data and the like. The processor 1010 deploys a program stored in the storage unit 1020 or the ROM 1030 into the RAM 1040, and executes the deployed program. The processor 1010 may be a central processing unit (CPU) or a graphics processing unit (GPU). The function of each unit in the server can be implemented by the processor 1010 executing the program, for example. The processor 1010 may include an internal buffer in which data and the like can be temporarily stored.

The computer described above is implemented by a computer system including a personal computer, a word processor, and the like. However, the present disclosure is not limited thereto, and the computer can be implemented by a server of a local area network (LAN), a host of computer (personal computer) communication, a computer system connected on the Internet, or the like. In addition, it is also possible to distribute functions to each device on the network and implement the computer with the entire network.

Note that the present disclosure is not limited to the example embodiments described above, and can be appropriately changed without departing from the concept. For example, the second example embodiment and the third example embodiment may be combined. That is, the model generation units 304a and 304b may generate the first physical condition estimation model and the second physical condition estimation model for each patient. Then, the physical condition information generation units 307a and 307b may generate the physical condition related information based on the estimated state information output from the first physical condition estimation model and the physical condition related information output from the second physical condition estimation model. In addition, the output control units 308a and 308b may output two types of physical condition related information to the patient terminal 100.

When generating the first physical condition estimation model and the second physical condition estimation model for each patient, the model generation units 304a and 304b may use the parameters of one physical condition estimation model in an optimization process for the parameters of the other physical condition estimation model. As a result, the optimization process can be speeded up.

Furthermore, in the above description, the output request process and the display process by the patient terminal 100 are performed on the application, but it is not essential that the processes function on the application.

In the above description, the estimation model DBs 305 and 305b stores the first or second physical condition estimation model for each patient ID, and the physical condition information generation units 307a and 307b read the first or second physical condition estimation model corresponding to the target patient. Alternatively, the estimation model DBs 305 and 305b may store the parameters of the first or second physical condition estimation model for each patient ID, and the physical condition information generation units 307a and 307b may read the parameters of the first or second physical condition estimation model corresponding to the target patient. In this case, the physical condition information generation units 307a and 307b generate the physical condition related information by using the first or second physical condition estimation model to which the read parameters are applied.

### REFERENCE SIGNS LIST

1, 1a INFORMATION PROCESSING SYSTEM
10 PATIENT SYSTEM
20 HOSPITAL SYSTEM
100 PATIENT TERMINAL
110 CAMERA
200 HOSPITAL TERMINAL
210 CAMERA
300, 300b INFORMATION PROCESSING APPARATUS (SERVER)
301, 301a REGISTRATION UNIT
302 LEARNING DB
3020 PATIENT ID
3021 REFERENCE IMAGE
3022 STATE INFORMATION
304, 304a, 304b MODEL GENERATION UNIT
305, 305b ESTIMATION MODEL DB
3051 PATIENT ID
3052a FIRST PHYSICAL CONDITION ESTIMATION MODEL
3052b SECOND PHYSICAL CONDITION ESTIMATION MODEL
306, 306b INFORMATION ACQUISITION UNIT
307, 307a, 307b PHYSICAL CONDITION INFORMATION GENERATION UNIT
308, 308a, 308b OUTPUT CONTROL UNIT
1000 COMPUTER
1010 PROCESSOR
1020 STORAGE UNIT
1030 ROM
1040 RAM
1050 COMMUNICATION INTERFACE
1060 USER INTERFACE
P PATIENT

What is claimed is:

1. An information processing system comprising:
at least one memory storing instructions; and
at least one processor configured to execute the instructions to:
acquire, for each patient, a reference image obtained by photographing a patient and state information regarding at least one of a physical condition state or a progress status of the patient at a time of photographing;
generate, for each patient, a physical condition estimation model for estimating a physical condition of the patient based on the reference image and the state information;
generate information related to the physical condition of a target patient by inputting a photographed image or predetermined state information of the target patient to the physical condition estimation model for the target patient; and
output the information related to the physical condition of the target patient.

2. The information processing system according to claim 1, wherein the state information contains information regarding a status suggesting a progress of a disease.

3. The information processing system according to claim 1, wherein the reference image includes an image area of a target region to be used for case determination for the patient.

4. The information processing system according to claim 3, wherein the at least one processor is further configured to execute the instructions to determine the target region for each patient based on medical record information of the patient.

5. The information processing system according to claim 1, wherein
the physical condition estimation model includes a first physical condition estimation model that receives the photographed image of the patient as an input and outputs estimated state information of the patient, and
the at least one processor is further configured to execute the instructions to generate the information related to the physical condition of the target patient based on the estimated state information in a case where the photographed image of the target patient is input to the first physical condition estimation model for the target patient.

6. The information processing system according to claim 1, wherein
the physical condition estimation model includes a second physical condition estimation model that receives the predetermined state information as an input and outputs a simulation image of the patient, and
the at least one processor is further configured to execute the instructions to generate the simulation image of the target patient corresponding to the predetermined state information as the information related to the physical condition of the target patient by using the second physical condition estimation model for the target patient.

7. An information processing method comprising:
acquiring, for each patient, a reference image obtained by photographing a patient and state information regarding a physical condition state or a progress status of the patient at a time of photographing;
generating, for each patient, a physical condition estimation model for estimating a physical condition of the patient based on the reference image and the state information;
generating information related to the physical condition of a target patient by inputting a photographed image or predetermined state information of the target patient to the physical condition estimation model for the target patient; and
outputting the information related to the physical condition of the target patient.

8. A non-transitory computer readable medium storing a program for causing a computer to execute:
acquiring, for each patient, a reference image obtained by photographing a patient and state information regarding a physical condition state or a progress status of the patient at a time of photographing;
generating, for each patient, a physical condition estimation model for estimating a physical condition of the patient based on the reference image and the state information;
generating information related to the physical condition of a target patient by inputting a photographed image or predetermined state information of the target patient to the physical condition estimation model for the target patient; and
outputting the information related to the physical condition of the target patient.

9. The information processing system according to claim 1, wherein
the first and physical condition estimation model is configured to be generated by using machine learning, and
the information related to the physical condition of the target patient is information for the target patient to make a decision about the physical condition.
